(19) United States
(12) Patent Application Publication
(10) Pub. No.: US 2023/0357958 A1
(43) Pub. Date: Nov. 9, 2023

(54) MARKING SILK PRODUCTS

(71) Applicant: SECURITY MATTERS LTD., D.N. Revel Eilot (IL)

(72) Inventors: Yifat BAREKET, Reut (IL); Hagit SADE, Ramat-Gan (IL); Or LIVERTZ, Ramla (IL); Dana GASPAR, Tel Aviv (IL); Michal FIRSTENBERG, Rehovot (IL); Nataly TAL, Nes Ziona (IL); Mor KAPLINSKY, Herzelia (IL); Haggai ALON, Kibbutz Naan (IL); Ron DAFNI, Tel Aviv (IL); Chen NACHMIAS, Tel Aviv (IL); Nadav YORAN, Tel Aviv (IL); Michal BURCK ZALTZMAN, Tel Aviv (IL)

(21) Appl. No.: 18/040,304

(22) PCT Filed: Aug. 3, 2021
(86) PCT No.: PCT/IL2021/050938

§ 371 Date: Feb. 2, 2023

Related U.S. Application Data
(60) Provisional application No. 63/129,269, filed on Dec. 22, 2020.
(60) Provisional application No. 63/062,269, filed on Aug. 6, 2020.

(51) Int. Cl. D01B 7/02; D01C 3/02; A01K 67/04; A23K 50/90

(52) U.S. Cl. CPC D01B 7/02; A01K 67/04; A23K 50/90; D01C 3/02

(57) ABSTRACT

A process is provided for identifying a production and/or commercial history of a silk fiber or a product made therefrom.

### TECHNOLOGICAL FIELD

The present invention is in the field of marking, tracing and managing a supply chain of silk and silk products.

### BACKGROUND

Silk production dates back thousands of years. Silk is regarded one of the most valuable raw materials in the fabrics industry. The production of silk yarn and silk fabric is a long and labor-intensive process.

The cultivation of silkworms for the purpose of silk producing is called sericulture. Sericulture involves raising healthy eggs through the chrysalis stage when the worm is encased in its silky cocoon. The chrysalis inside is destroyed before it can break out of the cocoon so that the precious silk filament remains intact. The healthiest moths are selected for breeding, and they are allowed to reach maturity, mate, and produce more eggs. Generally, one cocoon produces between 1,000 and 2,000 feet of silk filament, made essentially of two elements. The fiber, called fibroin, makes up between 75 and 90% of the fiber content, and sericin- a gum secreted by the caterpillar to glue the fiber into a cocoon, is present in an amount that is between 10 and 25 wt% of the fiber.

Silkworms feed mainly on leaves of the mulberry tree or on commercially available industrial feed. The mulberry leaves are fed to the voracious silkworms every few hours for a period of about a month. During this period the silkworms increase in size and shed their molt several times during that period. By twisting their head, the silkworms prepare to spin their cocoons. The worms spin a double strand of fiber in a figure-eight pattern and construct a symmetrical wall around themselves. The filament is secreted from each of two glands called spinneret located under the jaws of the silkworms. The resuk is a raw silk fiber, called bave.

The caterpillar spins a cocoon encasing itself completely. It can then safely transform into the chrysalis, which is the pupa stage. To avoid damage to the silk, which may result if the chrysalis breaks through the protective cocoon and emerge as moths, the chrysalis are destroyed by heat, typically by stoving.

Following stoving, the cocoons are soaked in hot water to loosen the sericin. Reeling of the silk fibers then proceeds, forming long and continuous silk threads. The reeled raw silk fibers are packaged into bundles, so-called books, and transported to manufacturing centers where the silk fibers are further processed.

In a process of degumming the remaining sericin is removed by soaking the silk fibers in soapy water. This results in a soft, white silk fiber which may be further processed and dyed to produce a variety of silk-based products.

Publication [1] disclosed functionalized silk fibroin security marker comprising one, two, three or more different security taggants selected from the group consisting of metallic particles, preferably metallic nanoparticles; magnetic particles, preferably magnetic nanoparticles; and peptide sequences, use of the security marker within the substrate and/or on the surface of a security document.

Publication [2] discloses loaded silk fibroin fibers as security features. The security fiber comprises silk fibroin loaded with a fluorescent chromophore or IR absorbed chromophore.

### PUBLICATIONS

EP 3 282 042
US 2016/0325579

### GENERAL DESCRIPTION

As indicated above, the process of silk manufacturing has changed relatively little over the centuries. A typical silk production process comprises sericulture, degumming of the silk fibers (in one or more repeated steps), reeling the silk fibers and processing thereof to produce a silk product that is optionally dyed.

The inventors of the technology disclosed herein have developed a process for marking silk fibers with an identifiable encoding marker that can distinguish one silk producer from the other, but which can also record the history of the particular silk product. By allowing silkworms to feed on leaves marked with a marking formulation, as disclosed herein, or by treating cocoons or fibers produced therefrom with a marking formulation, at any stage of the processing scheme mentioned above, under conditions that do not introduce any change to the silk processing steps (and thus do not have any effect on the resulting processed silk), and also under conditions which securely and irreversibly embed the markers within the silk fibers, detection of the marker, at any stage thereafter even in a finished marketable silk product, allows for authenticating a silk product and determining its processing and marketable history.

The markers may be detected and their concentration measured using a suitable reader throughout the production process of the silk fibers and the final product made therefrom. The ability to introduce a marker at any stage of the process such that its presence may be detected at any stage thereafter, renders the marking technique of the invention especially unique for encoding into the silk latent information such as the origin of the silkworms (that is, the farm from which the silkworms originated or sericulture farm), various dates of production, the processing facilities, the supplier or distributer of the silk fibers, the grade of the silk and so on. Thus, by having the ability to mark or encode a silk fiber or a product made therefrom, at any stage of processing, and at any stage thereafter, the production history may be latently embedded within the final product.

The “silk fiber” referred to herein may be any natural protein fiber that is composed of fibroin and is produced by insects larvae to form cocoons. Within the context of the present application the term mainly refers to fibers obtained from the cocoons of the larvae of the mulberry silkworm Bombyx mori cultivated in captivity.

The marking of the fibers or fabrics made therefrom according to the present invention may be carried out at any stage along the production process of the silk. The steps involved in silk production which may be modified to comprise a step of marking according to the invention are generally the following:
Silkworm feeding stageleaves of the mulberry tree or any other leaves may be coated or otherwise treated with a marking formulation prior to feeding. The marker elements or materials are taken up by the worms and secreted in the filaments making up the bave (the raw silk fiber);
-Bave treatment;
-Destroying the chrysalis by heat, typically by stoving;
-Soaking the cocoons in hot water to loosen the sericin;
-Reeling the silk fibers to form long and continuous silk threads:
-Degumming the fibers to remove the remaining sericin:
-Ennobling of the silk fibers;
-Processing of the fibers to yield the silk product, which may involve degumming, dyeing and ennobling phases:

A summary of silk processing and the inclusion of the various steps of marking the silk is depicted in FIG. 1.

Thus, in a process for manufacturing silk from silkworms, the process comprising feeding silkworms with leaves treated with a formulation comprising at least one XRF-identifiable marker or with a feeding composition comprising the marker (an industrial feeding composition for silkworms that is enriched with a marker) under conditions permitting said marker to be taken up by the worms, and/or treating a bave during the bave treatment stage, and/or cocoon and/or a silk fiber during the degumming stage and/or at any other stage with a formulation comprising the marker.

The invention also provides a process for identifying a silk, the process comprising feeding the silkworms or treating a cocoon thereof or a silk fiber with a formulation comprising at least one XRF-identifiable marker under conditions permitting embedding said marker in the silk fiber; and analyzing the presence of the XRF-identifiable marker in said fiber or product made therefrom, to thereby identify the silk. The analysis may be carried out as disclosed herein.

The XRF-identifiable marker is selected to indicate a particular property or information relating to the processed silk and may be thereafter unequivocally identified and monitored. Where the silk fiber is treated more than once with different marker formulations, as defined herein, each of the marker formulations may be configured to provide a latent marking that identifies a different property or information. Additionally, the concentration of the marker can also be measured enabling the encoding of information by associating different codewords for different concentrations of markers. Generally speaking, in silk production, marking may be used to identify any one or more of the following:
-origin of the silkworm;
-the farm where the silkworms were grown;
-the processing facility;
-date of processing;
-the processing protocol;
-the supplier of the bave or reeled silk; and
-other information relating to operational and logistical data such as batch, year, week, factory, operator, factory etc and marketing data, including customer, distributer, collection, and information relating to the supply chain of silk.

By enabling such latent encoding, identification and monitoring of the silk at any stage of processing, and even in a finished product, becomes possible.

A system for managing a supply chain of silk and silk products may include a database system (central or distributed) where data relating to silk and their marking is stored. For example, the database system may include information relating to the origin of the silk, the manufacturer of the silk produced from the worms, a batch of silk, the silk fibers, type of silk products as well as distributers and buyers. For that purpose, the device reading the marking (e.g. an XRF analyzer) may communicate with the database system. The database system may be an on-the-premises, cloud-based system or a distributed ledger. In an example, the database system may be a distributed blockchain system wherein a plurality of parties store and access the relevant data. In such a blockchain system a plurality of parties (for example, parties which are members of the same supply chain) may store and access data wherein the data stored is immutable, easily verifiable and, due the distributed design, inherently resistant to modification. In an example, the parties to the blockchain system may include farms, production facilities, suppliers, delivery companies, and even end users. In an example the marking on a silk fiber is read (detected) by a suitable XRF device and recorded every time it changes hands along the supply chain and recorded (e.g. automatically) on the blockchain allowing each party to easily verify the provenance and complete history of the silk and/or product made therefrom. Blockchain systems that are suitable for managing a supply chain of marked objects and products are described in International Pat. Applications PCT/IL2018/050499 and PCT/IL2019/050283 or any US applications derived therefrom, which are incorporated herein by reference.

The invention further provides a process for identifying a production and commercial history of a silk product, the process comprising
-feeding silkworms or treating a silkworm cocoon or a silk fiber produced therefrom with a formulation comprising a first XRF-identifiable marker at a first stage, under conditions permitting embedding said first marker in the silk fiber or bave;
-treating the silk fibers at a second stage with a second XRF-identifiable marker under conditions permitting embedding said second marker in the fibers; and
-analyzing the presence of the first and second XRF-identifiable markers in said silk product or silk fiber, to thereby determine the production and commercial history of the silk fiber or product made therefrom.

In another process the fiber is marked in a stage of destroying the chrysalis by heat, or in a hot water bath used for loosening sericin or in a stage of degumming, or at any stage prior to reeling of the silk fibers. The process may thus comprise:
-treating the cocoons or bave with a formulation comprising a first set of one or more XRF-identifiable markers under conditions permitting embedding said first marker in the fibers; wherein the first marker encoding is indicative of e.g., at least one parameter relating to the facility or process conditions. For example, the first set of markers may encode the farm from which the silkworms originated, the processing facility, and the grade/quality of the silk and so on;
-during reeling treating the silk fibers with a formulation comprising a second set of one or more XRF-identifiable markers under conditions permitting embedding said second set of markers in the fibers; wherein the second set of markers encoding is indicative of e.g., at least one parameter relating to the grade of the silk after undergoing treatment, batch number and so on;
after stage of reeling treating the silk fibers with a formulation comprising a third set of one or more XRF-identifiable markers under conditions permitting embedding said third set of markers in the fibers: wherein the third set of markers encoding is indicative of e.g., at least one parameter relating to the destination of the fibers, the intended further processing and so on;
-optionally during ennobling treating the silk fibers with a formulation comprising a further set of one or more XRF-identifiable markers under conditions permitting embedding said further set of markers in the fibers; and
-analyzing the presence of the first and/or second and/or third and/or optionally further sets of XRF-identifiable markers in said silk or in a product manufactured therefrom to determine one or more of the production or commercial history of the fiber or product made therefrom.

Generally speaking, the conditions used to embed the marker in the fibers are those used in the silk processing steps. No special conditions are utilized. This supports the uniqueness of processes of the invention whereby none of the processing conditions need to be modified to allow suitable and effective marking of the fibers.

In some embodiments, the silk is marked by marking the cocoon using a marking formulation; namely by immersing the cocoon in a water-based solution which includes one or more marking compositions, wherein each marking formulation includes one or more markers and possibly one or more additives.

In some embodiments, the silk is marked during the degununing process by adding one or more marking formulations, possibly with one or more additives, to the water-based solution used in the degumming process.

In some embodiments, the silk is marked by utilizing the dyeing process; namely, by adding one or more marking formulations to the water-based solutions used in the dyeing process.

At any stage of the process, by treating the silk as proposed herein, the marker becomes embedded or chemically associated or trapped within the silk to produce a substantially irreversible interaction with the marker. Markers which are applied to the silk at any stage can be detected after preparatory stages of production (i.e. all production processes prior to reeling) and after the silk fibers have undergone post treatment and dyeing. The markers can also be read from the finished silk after production, and even from a final silk product.

Any of the marking steps involves treating the cocoon, the bave, the fiber with a formulation comprising at least one XRF-identifiable marker under conditions permitting embedding said marker in the cocoon, bave or fiber. The term “treating” or any lingual variation thereof involves contacting with the formulation, by any way available in the art. Typically application is by continuous washing or spraying with or soaking in a water-based formulation or solution, or coating, padding with a water-based formulation or solution, which includes one or more markers, herein the “marker formulation”. Apart from the marker molecules or marker elements, the marker formulation may also include processing agents such as surfactants, catalysts and enzymes: and intermediate or bridging agents that are capable of chemically associating the marker to a region, a material or an atom of the silk.

Any silk fiber, processed or provided in any natural form, c.g., bave, cocoon unreeling fibers, as well as processed and reeled fibers may be treated once with a marker formulation or may be treated with two or more same or different marker formulations, at different stages of the silk production process, wherein each of the two or more marker formulations may contain the same or different markers. By enabling consecutive marking sessions, each of the silk fibers may be encoded with a variety of important information relating to origin, date of processing, site of processing, manufacturer, etc. For example, a first marking session involves feeding the silkworms with leaves treated with a marking formulation, before silk production and then again during silk processing and/or during the dyeing or the finishing stages of silk production. Surprisingly, notwithstanding the stage of application, the marker which is applied to the fibers, even at the initial stage prior to production, remains embedded in or on the silk fibers throughout the production process and may be read even from the final finished silk product.

The marker or marking formulation comprises at least one XRF-identifiable marker. The marker may be detected and measured by X-Ray fluorescence (XRF) spectrometers (readers) which may detect and identify their response (signature) signals. In an example, the XRF readers are Energy Dispersive X-Ray fluorescence EDXRF spectrometers. XRF markers are flexible, namely, they may be combined, blended or form compounds with, huge range of carriers, and materials.

The marker is typically water soluble, permitting facile and effective marker penetration into the silk fiber. However, where the marker is water-insoluble, the aqueous formulation may comprise the marker in suspended or dispersed forms. The marker may be in a form of a metal atom, a metal oxide, or a metal salt such as a metal sulfide, a metal carbonate, perchlorate, chlorate, nitrate, hydroxides, sulfates, sulfites, phosphates, chromates, oxalate and others; or in the form of an organometallic or an organohalide material. The organometallic material may be selected amounts organic anions that are ionically associated with at least one metal atom (metal cation). Non-limiting examples include metal phenolates, metal acrylates, metal-associated anilines and others. The organohalide is at least one organic material substituted with at least one halide e.g., bromide, iodine, chloride. Such organohalides include halide-substituted phenols, halide substituted anilines, halide-substituted epoxies, halide-substituted acrylates, halide-substituted amides, halide-substituted acids, halide-substituted glycols and others.

Notwithstanding the type of marker, the marker is an atom or a material that is not present in the silk fibers; nor in any of the processing solutions typically used in silk production. Using a marker that is not native to the silk or the process enables accurate and confident encoding and further generating a complex encoding scheme. Atoms or materials which may be present in the silk or involved in the manufacturing process and which may be regarded as XRF-identifiable contribute nothing to the ability contemplated herein to determine the production and commercial history of a silk-based product as such native or accidental materials do not constitute a code for determining product or conunercial history. The code relies only on a material added in a predefined concentration, composition and optionally in combination with one or more additional marker (atom or material). Thus, as used herein, the XRF-identifiable marker is one which is present in a marker formulation and is actively added or used for the purposes disclosed herein.

The marker may be any atom of the periodic table. The atom may be provided as a metal salt, a complex, an organic molecule or an inorganic molecule. Where the marker is a metal or a metal containing material, e.g.. organometallic material the metal atom may be selected from aluminum (provided as e.g., aluminum sulfate), titanium (provided as, e.g.. titanium sulfate), cobalt (provided as e.g.. cobalt nitrate hydrate, cobalt gluconate, cobalt glycinate), nickel (provided as nickel nitrate hydrate, nickel glycinate), yittrium (provided as e.g., yttrium nitrate), cadmium (provided as e.g., cadmium nitrate), tin (provided as e.g., tin chloride), scandium, titanium, niobium, silver, tungsten, zinc, zirconium, manganese, barium, calcium, chrome, iron, gallium, germanium, lanthanum, magnesium, selenium, strontium, terbium, copper, lead, molybdenum, vanadium, bismuth, antimony, tantalum and cesium (provided as e.g., cesium carbonate).

Other metal-based markers may be provided in a water-insoluble form. Such include aluminum oxide, scandium acetate, titanium oxide, cobalt acetyl acetonate, cobalt carbonate, cobalt dibromo, nickel acetyl acetonate, nickel acrylate, yttrium oxide, niobium oxide, silver carbonate, silver chloride, tin ethyl hexanoate, tungsten oxide and others.

Halide-based markers include tri-iodine phenol (TIP), tribromophenol (TBP), tri chlorophenol (TCP), 2,2-bis(bromomethyl) propane-1,3-diol, 2,4,6-tribromo aniline, pentabromobenzyl acrylate, 4,5,6,7-tetrabromoisobenzofuran-1,3-dione, ammonium bromide and others.

Excluded are atoms of materials used in an industrial silk manufacturing process or which are naturally present in the silk fibers.

The invention further provides a fibroin-rich silk fiber, or generally a silk fiber, produced from a silk-producing animal, the fiber being associated, embedded or adsorbed with at least one XRF identifiable marker, as disclosed herein.

The invention further provides a silk product comprising at least one silk fiber of the invention.

Also provided is a product, such as a fabric, comprising at least one silk fiber of the invention.

A reading unit may be used for detecting the marking compositions and/or measuring their concentrations or relative concentration in the preselected areas or complete area on the surface of the object. In an example the marking composition includes markers which arc identifiable by XRF analysis and the verification unit comprises an XRF analyzer which emits an X-ray or Gamma-ray radiation towards the object and detects the X-ray signal (a response signal) that is emitted from the markers in response. Such an XRF analyzer may be configured to measure/estimate the concentration or relative concentration of each of the markers according to the detected response signal. The concentrations of the markers may be indicative of the information encoded by the marking composition on the object. Accordingly, based on the measured/estimated concentration the system may be configured and operable to verifying that the applied marker composition indeed matches/encodes the intended information/authentication data that should have had being marked on the object and possibly also verifies the quality of the marking applied by the marking device (i.e. the quality may be determined based on the signal to noise (SNR) of the detected signal).

Thus aspects and embodiments of the present invention provide:

A process for marking silk manufactured from silkworms with an XRF-identifiable marker, the process comprising:
-feeding silkworms with leaves treated with a formulation comprising at least one XRF-identifiable marker or feeding silkworms with a feeding composition enriched with a formulation comprising at least one first XRF-identifiable marker under conditions permitting said marker to be taken up by the silkworms, and/or
-treating a have with a formulation comprising at least one XRF-identifiable marker, during the bave treatment stage, and/or
-treating a cocoon with a formulation comprising at least one XRF-identifiable marker, and/or
-treating a silk fiber during a degumming stage or during an ennobling stage to thereby mark the bave, the cocoon, and/or the silk fiber with the at least one marker, wherein the XRF-identifiable marker is not a native material to a silk fiber or silk manufacturing process.

A process for identifying a silk, the process comprising marking the bave, cocoon or silk according to the process of the invention and analyzing the presence of the XRF-identifiable marker in a silk fiber or a product made from said bave, cocoon or silk fiber, to thereby identify the silk.

A process of the invention, wherein the XRF-identifiable marker is selected to identify a property or information relating to the processed silk.

A process of the invention, wherein the silk fiber is treated more than once with different marker formulations, each of the marker formulations being configured to provide a latent marking that identifies a different property or information.

A process of the invention, wherein the marker is configured to identify any one or more of:
-origin of the silkworm;
-the farm where the silkworms were grown;
-the processing facility;
-date of processing;
-the processing protocol;
-the supplier of the have or reeled silk; and/or
information relating to operational and logistical data.

A process for identifying a production or commercial history of a silk product, the process comprising
-feeding silkworms or treating a silkworm cocoon or a silk fiber produced therefrom with a formulation comprising a first XRF-identifiable marker at a first stage, to embed said first marker in the silk fiber or bave:
-treating the silk fiber at a second stage with a second XRF-identifiable marker to cmbed said second marker in the silk fiber; and
-analyzing the presence of the first and second XRF-identifiable markers in said silk fiber or product made therefrom to thereby identify the production or commercial history of the silk fiber.

A process of the invention, wherein the silk fiber is marked during a stage of destroying chrysalis, or during a stage of loosening sericin or during a stage of degumming.

A process of the invention, wherein the process comprising:
-treating the cocoon or bave with a formulation comprising a first set of one or more XRF-identifiable markers to embed said first marker in the silk fiber, wherein the first set of markers encoding at least one parameter relating to the facility or process conditions;
-during reeling treating the silk fiber with a formulation comprising a second set of one or more XRF-identifiable markers to embed said second set of markers in the silk fiber; wherein the second set of markers encoding at least one parameter relating to the grade of the silk fiber after undergoing treatment;
after reeling treating the silk fiber with a formulation comprising a third set of one or more XRF-identifiable markers to embed said third set of markers in the silk fiber; wherein the third set of markers encoding at least one parameter relating to the destination of the silk fiber or an intended further processing;
-optionally during ennobling treating the silk fiber with a formulation comprising a further set of one or more XRF-identifiable markers; and
-analyzing presence of the first and/or second and/or third and/or further sets of XRF-identifiable markers in said silk or in a product manufactured therefrom to determine production or commercial history of the silk fiber or product made therefrom.

A process of the invention, wherein the analyzing step comprises directing an X-ray or Gamma-ray radiation towards the silk fiber or product made therefrom and detecting a response X-ray signal emitted from the marker in response, such that said response signal is indicative of presence, concentration or relative concentration of the marker to thereby provide information encoded by the marker on the production or commercial history of the silk fiber or product made therefrom.

A process of the invention, wherein the silk fiber is marked by immersing the cocoon in a water-based solution comprising one or more markers and optionally one or more additives.

A process of the invention, wherein the silk is marked by utilizing a dyeing solution comprising one or more markers.

A process of the invention, wherein the XRF-identifiable marker is in a form of a metal atom, a metal oxide, or a metal salt, or an organometallic or an organohalide material.

A process of the invention, wherein the marker is a metal or a metal containing material, the metal atom being optionally selected from aluminum, titanium, cobalt, nickel, yttrium, cadmium, tin, scandium, titanium, niobium, silver, tungsten, zinc, zirconium, vanadium, manganese, copper, lead, molybdenum, vanadium, bismuth, antimony, tantalum and cesium.

A process of the invention, wherein a metal-based marker is selected from aluminum oxide, scandium acetate, titanium oxide, cobalt acetyl acetonate, cobalt carbonate, cobalt dibromo, nickel acetyl acetonate, nickel acrylate, yttrium oxide, niobium oxide, silver carbonate, silver chloride, tin ethyl hexanoate, tungsten oxide and others.

A process of the invention, wherein a halide-based marker is selected from tri-iodine phenol (TIP), tribromophenol (TBP), tri chlorophenol (TCP), 2.2-bis(bromomethyl) propane-1,3-diol, 2,4,6-tribromo aniline, Penta-bromobenzyl acrylate, 4,5,6,7-tetrabromoisobenzofuran-1,3-dione and ammonium bromide.

A fibroin rich silk fiber produced from a silk-producing animal, the fiber being associated, embedded or adsorbed with at least one XRF-identifiable marker.

A silk product comprising at least one silk fiber according to the invention.

A fabric comprising at least one silk fiber according to the invention.

A system for managing a supply chain of silk and silk products, the system comprising a database system (central or distributed) comprising data relating to silk and their marking with an XRF-identifiable marker.

A process for identifying a production and/or commercial history of a silk fiber or a product made therefrom, the fiber or product having been marked with at least one XRF-identifiable marker, the process comprising directing an X-ray or Gamma-ray radiation towards the fiber or product made therefrom and detecting a response X-ray signal emitted from the marker in response, such that said response signal is indicative of presence, concentration or relative concentration of the marker to thereby provide information encoded by the marker on the production or commercial history of the silk fiber or product made therefrom.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 depicts a process of the invention in accordance with some of its embodiemnts.

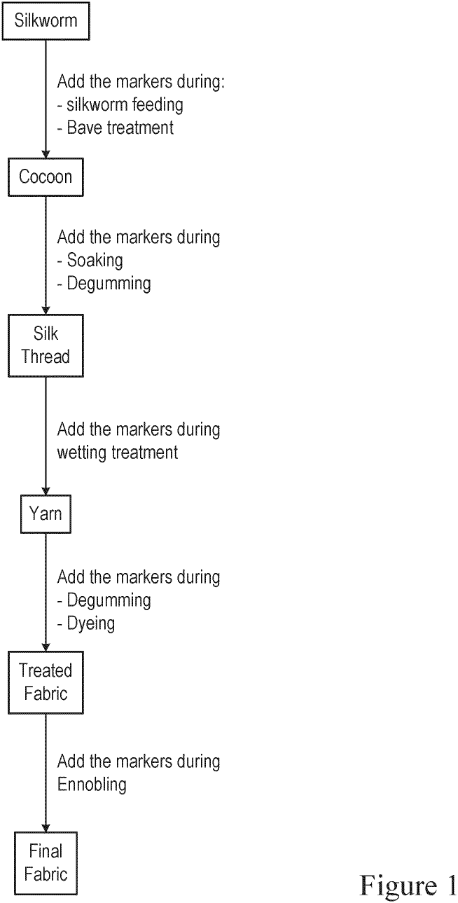

### DETAILED DESCRIPTION OF EMBODIMENTS

### Example 1

Silk used in the textile industry is produced by larvae of moth caterpillars, the best known of which is silk obtained from the cocoons of the larvae of the mulberry silkworm. The marking of the silk is obtained by adding a marking composition to the food consumed by the silk caterpillar. The silk fibers are produced by two glands and is naturally extruded from two openings (spinnerets) in the caterpillar’s head. By allowing the caterpillar to consume a one or more marking compositions the silk fiber produced by caterpillar may include or attached to the markers.

The marking compositions may for example include soluble or insoluble inorganic salts, oxides, substituted phenols, carbide, inorganic salts such as salt of sulfides, carbonates, bicarbonate, sulfate, phosphate, nitrate, acrylates, acetate, acetylacetonate, ethylexanoate, gluconate, glycinate and anilines. In an example, the dry food or consumable dry material is added to a water-based solution (marking solution) comprising one or more marking compositions. The solution with the dry material may be heated and then dried such that a solid consumable (for the caterpillar) material is obtained. The marked solid material is then fed to the caterpillar.

In another example, the caterpillar is allowed to consume the one or more marking compositions by applying it directly to the leaves, or plant-parts eaten by the caterpillar, such as mulberry leaves. In this example the one or more marking composition may be applied to the leaves by spraying, dripping, brushing or any method for applying a liquid solution the leaf surface. In another example, the leaves are marked by immersing the leaves in a marking solution comprising one or more marking compositions.

### Example 2

Silk is produced from the cocoon of the silkworm caterpillar by extracting continuous threads from the cocoon. This follows a process of degumming in which the cocoons are placed and boiled in a water-based solution in order to soften and dissolve the gum that is holding the cocoon together.

The cocoons are marked by a separate process in which the cocoons are immersed in a water-based marking solution. The marking solution includes one or more marking compositions each including one or more markers (e.g. materials and elements that may be detected by XRF reader). The solution may also include at least one of processing agents such as surfactants classified as anionic, cationic, zwitterionic polymeric and/or non-ionic with various HLB (hydrophilic- lipophilic balance), catalyst, enzymes, radical initiator and fixation agents; and intermediate or bridging agents comprising intermediate molecules which will bond the marking composition to proteins in the silk fiber.

The one or more marking compositions may include oxides, substituted phenols, carbide, inorganic salts such as salt of sulfides, carbonates, bicarbonate, sulfate, phosphate, nitrate, acrylates, acetate, acetylacetonate, ethylexanoate, gluconate, glycinate and anilines. The marking composition may also include organo-halides.

The intermediate molecules are utilized for attaching or bonding the marking composition and or/the marker elements to the silk fibers. Specifically, the intermediate molecules function as a bridge connecting the active residues in the amino acids comprising the proteins in the silk fiber to the marking molecules and/or elements within the marking composition. The intermediate molecules may bond with the Carboxylic, hydroxylic, sulfides and the amine active residues in the amino acids. In an example the amino acids are glycine, alanine, and serine. In an example the intermediate molecules may be aldehydes such as aromatic aldehydes. In a particular example, the intermediate molecules may be one of the following: polymer, polysaccharide and/or aldehydes and its derivatives.

The marking of the cocoon by a separate marking process may be carried out either prior to the degumming process or after the degumming process or both before and after the degumming process. Furthermore, it may be carried out as a separate process during all stages of silk production.

### Example 3

Silk worms were divided into two groups: a control group and a marked group which was fed on an artificial food:

(https://www.wwb.co.uk/index.php?route=product/product&keyword=silkworm&product_id=6174 ) containing marking materials according to the invention.

The marking materials were dissolved in water to a concentration of between 0.001% and 1% w/w the XRF identifiable atom. The marking materials were selected from AgNO₃, Ba(NO₃)₂, BiCl₃, NH₄Br, CdCl₂, Co(NO₃)₂•6H₂O, Cu(NO₃)₂·3H₂O, La(CH₃CO₂)₃ • xH₂O, MnSO₄ · H₂O, Na₂SeO₄ and others.

Mulberry leaves were soaked in a solution of the marker material and thereafter were fed to the worms. When dry food was used, it was absorbed with the marking solution to absorb the solution to 200% of its original weight and thereafter was fed to the worms. Feeding was over a period of 3 weeks.

After the cocoon was developed, three independent measurements were taken: 5 days after cocoon formation, and 10 days after cocoon formation. The empty cocoon was also measured.

The markers were read and identified in the cocoon in all cases.

### Example 4

Cocoons were added into a marker solution containing up to 2000 ppm of the XRF identifiable atom. The markers were selected as in Example 3 above. The ratio cocoon to solution was between 1:60 and 1:200.

For some of the solutions, functional materials such as chitosan or chitosan derivatives and others were added to assist association between the marker materials and the substrate. The amount of the functional material was not greater than 25 ppm or higher.

Typically, the cocoons were left in the solution at a temperature between 40 and 100° C. for a period between 4 and 10 minutes, to mimic many of the processing conditions used in silk processing.

For the degumming step, a soap solution containing 1% natural soap was prepared to a pH between 9 and 9.8. The cocoons were soaked in the solution for a period of about two hours at a temperature between 85 and 95° C. Thereafter the cocoons were removed from the solution, washed and dried.

A dyeing solution containing 0.01% a water soluble dye and 0.1% acetic acid was prepared. At a temperature between 75 and 90° C. the cocoons were soaked for a period of 1 hour.

At the end of the complete process, markers were read.

### Example 5

A wetting solution containing 5-15% of a material mixture was prepared. The material mixture contained one or more of softening agents, natural and synthetic waxes, Anionic and non-ionic emulsifiers, glycols, fatty acids having between 14 and 20 carbon atoms, natural esters and others. To the solution markers selected as in Example 2 were added at a concentration of 0.01 and 1% or between 0.002% and 0.03% w/w.

Cocoons were soaked in the solution at a temperature below 30° C. for a period of between several minutes and 1 hour. The cocoons were thereafter removed and dried.

Degumming and dyeing was carried out as in Example 4 and the markers were thereafter measured.

### Example 6

Marking of the silk may be performed during degumming by introducing one or more marking compositions (each including one or more markers, and optionally additional additives), into a solution used in the degumming process. In an example one or more soluble marking composition are added into the aqueous solution in which degumming is carried out. In another example one or more marking compositions together with at least one of processing agents (such as surfactants, enzymes, and catalysts) and a bridging agent comprising intermediate molecules.

### Example 7

In a similar fashion to solutions prepared as above, ennobling solutions were prepared and used. Marker formulations suitable for each of the following steps were prepared and tested to confirm the ability to mark and read the cocoon or silk fiber generated therefrom.

1. A process for marking silk manufactured from silkworms with an XRF-identifiable marker, the process comprising:
feeding silkworms with leaves treated with a formulation comprising at least one XRF-identifiable marker or feeding silkworms with a feeding composition enriched with a formulation comprising at least one first XRF-identifiable marker under conditions permitting said marker to be taken up by the silkworms, and/or
treating a bave with a formulation comprising at least one XRF-identifiable marker, during the bave treatment stage, and/or
treating a cocoon with a formulation comprising at least one XRF-identifiable marker, and/or
treating a silk fiber during a degumming stage or during an ennobling stage to thereby mark the bave, the cocoon, and/or the silk fiber with the at least one marker, wherein the XRF-identifiable marker is not a native material to a silk fiber or silk manufacturing process.

2. A process for identifying a silk, the process comprising marking the bave, cocoon or silk according to the process of claim 1 and analyzing the presence of the XRF-identifiable marker in a silk fiber or a product made from said bave, cocoon or silk fiber, to thereby identify the silk.

3. The process according to claim 1, wherein the XRF-identifiable marker is selected to identify a property or information relating to the processed silk.

4. The process according to claim 1, wherein the silk fiber is treated more than once with different marker formulations, each of the marker formulations being configured to provide a latent marking that identifies a different property or information.

5. The process according to claim 1, wherein the marker is configured to identify any one or more of:
origin of the silkworm;
the farm where the silkworms were grown;
the processing facility;
date of processing;
the processing protocol;
the supplier of the bave or reeled silk; and/or
information relating to operational and logistical data.

6. A process for identifying a production or commercial history of a silk product, the process comprising
feeding silkworms or treating a silkworm cocoon or a silk fiber produced therefrom with a formulation comprising a first XRF-identifiable marker at a first stage, to embed said first marker in the silk fiber or bave;
treating the silk fiber at a second stage with a second XRF-identifiable marker to embed said second marker in the silk fiber; and
analyzing the presence of the first and second XRF-identifiable markers in said silk fiber or product made therefrom to thereby identify the production or commercial history of the silk fiber.

7. The process according to claim 1, wherein the silk fiber is marked during a stage of destroying chrysalis, or during a stage of loosening sericin or during a stage of degumming.

8. The process according to claim 7, the process comprising:
treating the cocoon or bave with a formulation comprising a first set of one or more XRF-identifiable markers to embed said first marker in the silk fiber; wherein the first set of markers encoding at least one parameter relating to the facility or process conditions;
during reeling treating the silk fiber with a formulation comprising a second set of one or more XRF-identifiable markers to embed said second set of markers in the silk fiber; wherein the second set of markers encoding at least one parameter relating to the grade of the silk fiber after undergoing treatment;
after reeling treating the silk fiber with a formulation comprising a third set of one or more XRF-identifiable markers to embed said third set of markers in the silk fiber; wherein the third set of markers encoding at least one parameter relating to the destination of the silk fiber or an intended further processing;
optionally during ennobling treating the silk fiber with a formulation comprising a further set of one or more XRF-identifiable markers; and
analyzing presence of the first and/or second and/or third and/or further sets of XRF-identifiable markers in said silk or in a product manufactured therefrom to determine production or commercial history of the silk fiber or product made therefrom.

9. The process according to claim 8, wherein the analyzing step comprises directing an X-ray or Gamma-ray radiation towards the silk fiber or product made therefrom and detecting a response X-ray signal emitted from the marker in response, such that said response signal is indicative of presence, concentration or relative concentration of the marker to thereby provide information encoded by the marker on the production or commercial history of the silk fiber or product made therefrom.

10. The process according to claim 1, wherein the silk fiber is marked by immersing the cocoon in a water-based solution comprising one or more markers and optionally one or more additives.

11. The process according to claim 1, wherein the silk is marked by utilizing a dyeing solution comprising one or more markers.

12. The process according to claim 1, wherein the XRF-identifiable marker is in a form of a metal atom, a metal oxide, or a metal salt, or an organometallic or an organohalide material.

13. The process according to claim 12, wherein the marker is a metal or a metal containing material, the metal atom being optionally selected from aluminum, titanium, cobalt, nickel, yttrium, cadmium, tin, scandium, titanium, niobium, silver, tungsten, zinc, zirconium, vanadium, manganese, copper, lead, molybdenum, vanadium, bismuth, antimony, tantalum and cesium.

14. The process according to claim 12, wherein a metal-based marker is selected from aluminum oxide, scandium acetate, titanium oxide, cobalt acetyl acetonate, cobalt carbonate, cobalt dibromo, nickel acetyl acetonate, nickel acrylate, yttrium oxide, niobium oxide, silver carbonate, silver chloride, tin ethyl hexanoate, tungsten oxide and others.

15. The process according to claim 12, wherein a halide-based marker is selected from tri-iodine phenol (TIP), tribromophenol (TBP), tri chlorophenol (TCP), 2,2-bis(bromomethyl) propane-1,3-diol, 2,4,6-tribromo aniline, Penta-bromobenzyl acrylate, 4,5,6,7-tetrabromoisobenzofuran-1,3-dione and ammonium bromide.

16. A fibroin rich silk fiber produced from a silk-producing animal, the fiber being associated, embedded or adsorbed with at least one XRF-identifiable marker.

17. A silk product comprising at least one silk fiber according to claim 16.

18. A fabric comprising at least one silk fiber according to claim 16.

19. A system for managing a supply chain of silk and silk products, the system comprising a database system (central or distributed) comprising data relating to silk and their marking with an XRF-identifiable marker.

20. A process for identifying a production and/or commercial history of a silk fiber or a product made therefrom, the fiber or product having been marked with at least one XRF-identifiable marker, the process comprising directing an X-ray or Gamma-ray radiation towards the fiber or product made therefrom and detecting a response X-ray signal emitted from the marker in response, such that said response signal is indicative of presence, concentration or relative concentration of the marker to thereby provide information encoded by the marker on the production or commercial history of the silk fiber or product made therefrom.
